(19) United States
(12) Patent Application Publication
(10) Pub. No.: US 2025/0017522 A1
(43) Pub. Date: Jan. 16, 2025

(54) SYSTEM AND METHOD FOR NON-INVASIVE MEASUREMENT OF MECHANICAL PROPERTIES OF CORTICAL BONE

(71) Applicant: CGK Technologies LLC, Columbus, OH (US)

(72) Inventor: William D. Timmons, Columbus, OH (US)

(73) Assignee: CGK Technologies LLC, Columbus, OH (US)

(21) Appl. No.: 18/714,622

(22) PCT Filed: Dec. 1, 2022
(86) PCT No.: PCT/US22/80771

§ 371 Date: May 30, 2024

Related U.S. Application Data
(60) Provisional application No. 63/285,974, filed on Dec. 3, 2021.

(51) Int. Cl. A61B 5/00

(52) U.S. Cl. CPC A61B 5/4504; A61B 5/0051; A61B 5/4884; A61B 5/702

(57) ABSTRACT

A system and method for determining mechanical properties of a bone of an individual is disclosed. The mechanical properties of the bone, for example, left ulna are determined through the imposition of constraints on the estimation of model parameters and combinations of model parameters of the tissue response when using mechanical response tissue analysis (MRTA) and its derivatives such as cortical bone mechanics technology (CBMT), when spurious modes of vibration are present in the data, and in individuals where soft tissue effects are large such as in those persons with high body mass index (BMI) and/or muscularity. The system comprises a test probe unit having a mounting system, a test probe with sensors, a vibrator unit having a vibrator motor, and a controller having a processor and a memory containing computer readable and executable instructions which are executed by the processor to perform multiple functions.

### CLAIM OF PRIORITY UNDER 35 U.S.C. § 119

The present application for patent claims priority to U.S. Provisional Application No. 63/285,974 entitled “SYSTEM AND METHOD FOR NON-INVASIVE MEASUREMENT OF MECHANICAL PROPERTIES OF CORTICAL BONE” filed Dec. 3, 2021, which is assigned to the assignee hereof and hereby expressly incorporated by reference herein.

### TECHNICAL FIELD

The invention disclosed herein generally relates to in-vivo determination of bone and soft tissue properties such as stiffness, damping, and participating masses, in response to mechanical perturbations applied to the overlying soft tissue. More particularly, the invention disclosed herein relates to systems and methods for determining the properties of bone and surrounding soft tissues through the optimal imposition of constraints on parameters and combinations of parameters of a model fit to the tissue response when using mechanical response tissue analysis (MRTA) and its derivatives such as cortical bone mechanics technology (CBMT) when spurious modes of vibration are present in the data and in individuals where soft tissue effects are large, i.e., persons having high body mass index (BMI) and/or muscularity.

### BACKGROUND

Mechanical Response Tissue Analysis (MRTA) is a non-significant risk, non-invasive, radiation-free, vibration analysis technique for making immediate, direct functional measurements of the bending stiffness of long bones in humans in vivo. In MRTA, a vibratory excitation is applied to the skin overlying cortical bone (typically the ulna or the tibia) of a braced or restrained body part (typically forearm or lower leg) while force and acceleration are measured at the interface between the excitatory signal and the skin. The force and acceleration data are then used to calculate a frequency response function H(jw) that describes the behavior of the mechanical system. The poles and zeros of a generalized transfer function PZ(jw) (the PZ model) are fit to H(jw) in some embodiments of MRTA (for example, by using a system analyzer such as the HP3562A), while in other embodiments the coefficients of a generalized transfer function TF(jw) (the TF model) are iteratively estimated until TF(jw) converges to an acceptably close fit to H(jw). In yet other embodiments, the inverse TF or PZ models are fit to the inverse frequency response function.

In either case, the coefficients are then converted algebraically to the form of a biomechanical (BM) model of skin, bone, and surrounding soft tissue, and sometimes the supports. The BM model includes a spring constant of the bone (the transverse bending stiffness K_{b}), which is then used to calculate the flexural rigidity EI of the bone based on the bone's measured length L_{b} using the formula EI=K_{b}L_{b}³/48.

Other parameters common in the BM models includes the bone damping factor Bb, which reflects the ability of the bone to absorb and dissipate energy, the participating mass of the bone, M_{b} (according to Steele et al. 1988, M_{b} is equivalent to approximately half the bone mass), and similar parameters for the overlying tissues. BM models diverge from here, as there may be terms for peripheral tissues and end supports. For the remainder of this disclosure, the 7-Parameter Model used by Steele (1991) and later researchers (e.g., Bowman & Loucks, 2019b) is assumed as the prototypical BM model, though those skilled in the art will realize that the methods and systems disclosed here also apply to other BM models as well.

A major problem with MRTA is that the derivation of the BM model assumes the presence of only the first mode of vibration in the axial (vertical) direction. However, most bones are not uniform beams, and their cross-sectional profiles vary along their length. Therefore, higher, lateral, and/or torsional modes of vibration will be present in the vibrations. Additionally, the mechanical behavior of the bone's end supports can contribute to the overall perturbation response. These problems can lead to TF and PZ model parameter estimates that violate the BM model assumptions. Common attempts at solving these problems include (a) repeat measurements at different angles and/or different locations along the length of the body part, followed by some form of outlier rejection and averaging, and (b) addition of more elements into the biomechanical model. The first approach adds more complexity and time to the data collection process, making it highly dependent on operator skill and more costly to operate, while the latter approach adds degrees of freedom to the model parameter estimation process, which can affect confidence in the model parameter estimates. This is especially true when noise is present and when the perturbation data are attenuated by larger amounts of soft tissue, such as in individuals with higher BMI and/or higher muscularity. Both noise and attenuation lower the signal to noise ratio in the collected data and thereby decrease confidence in the model parameter estimates, often severely.

Similar to the repeat measurement approach, cortical bone mechanics technology (CBMT, or “Bowman's Method”) repeats MRTA measurements at discreet positions in a transverse direction across the body part as described in U.S. Pat. No. 10,299,719B2 (Bowman et al., 2019a), Bowman and Loucks (2019b), Bowman and Loucks (2020), and US2021/0045636A1 (Bowman and Loucks, 2021). In CBMT, the measurement system reaches a “sweet spot” as it transits across the skin over the bone, wherein the bone bending becomes primarily (though not entirely) axial (vertical) in direction and primarily (though not entirely) consists of the first mode of vibration. At the sweet spot, estimates of K_{b} more closely represent the transverse bending stiffness of the bone since fewer non-primary and off-axis modes of vibration are present.

However, even though CBMT reduces higher, lateral, and/or torsional modes of vibration at the sweet spot, these modes are still present and can distort the model parameter estimates. Without correcting for these distortions, CBMT often fails to produce accurate results in all but ideal body types. In an attempt to avoid fitting the model to unwanted modes of vibration, a subsequent modification to CBMT resorts to repeatedly fitting the TF model to an extensive set of frequency subspaces until a set of BM model parameters (algebraically calculated from the TF model parameter estimates) are found that satisfy a list of rejection and acceptance criteria. As a result, much data is disregarded leaving less information available for each model fit and thereby increasing the uncertainty (and randomness) in the parameter estimates. In addition, because the search is exhaustive, many model fits are generated so that any attempt to select a “best” model fit leads to a high chance for type I error (the acceptance of a defective model fit) for all but ideal body types in low-noise settings. Furthermore, the signal attenuation problem described above for individuals with larger amounts of soft tissue, such as those with higher BMI and/or higher muscularity, remains for CBMT, so that it too often suffers from severely decreased confidence in the model parameter estimates. The soft tissue attenuation effect is especially exacerbated when combined with CBMT's frequency subspace search method's inherently low signal to noise ratio due to significant amounts of disregarded data.

Another key source of errors in MRTA and its derivatives like CBMT is numerical instability caused by poorly conditioned data and/or ill-conditioned model conversions. Numerical instabilities, especially when combined with the other error sources, can produce erroneous model parameter estimates, so that MRTA and its derivatives like CBMT often fail outright, produce poor estimates of EI and related metrics, misidentify the bone and skin peaks, and/or estimate intermediate (PZ and/or TF) model parameters that correspond to negative or complex (having an imaginary component) values for multiple terms in the BM model including K_{b}. When these last two problems occur, the BM model becomes nonsensical.

The imposition of known or supplementary information in the form of constraints on the model parameters during their estimation can overcome many of these long-standing problems and yield better estimates. For example, to prevent the BM model parameters from becoming nonsensical as discussed immediately above, constraints can be imposed on the PZ and TF model parameters such that the roots of the quadratic equation in Eq. 11 are real-valued, and such that the BM model parameters themselves are nonnegative. Thus, the imposition of constraints can optimally force “reasonableness” onto the parameter estimates. In addition, constraints can reduce the degrees of freedom, thereby increasing confidence in the parameter estimates.

Because the BM model form attempts to model the physics underlying the body part being analyzed, including the masses, stiffnesses, and damping factors of the bone and tissues of interest, and in some cases, the end supports, the BM model provides a natural framework for developing physically meaningful and relevant constraints on the parameters of the model. The TF and PZ models, on the other hand, provide a natural framework for developing other types of useful constraints, such as overall system stability, settling time, resonances, etc. Thus, each model form can be used as a natural framework for deriving constraints that impose known information. However, while TF, PZ, and BM model forms can be readily interchanged, constraints that are linear and convex in one model space (and thus easily applied in that model space) may not be linear and convex when translated to another model space. For example, the BM model parameters map to multiple TF model parameters in a straightforward way as shown in Eq.'s 2-8, but the mapping is nonlinear, making a set of constraints that impose simple bounds on BM model parameters difficult to convert to similarly simple constraints on the TF model parameters. In addition, one or more of the converted constraints are likely to be non-convex, creating further complexities when optimally imposing such constraints on the estimation of TF or PZ model parameters. A useful approach here is to divide the constraints into hard and soft categories, where a hard constraint is defined as a constraint that cannot be violated, and a soft constraint is defined as a constraint that can tolerate some violation of the condition. Soft constraints can be implemented as penalty functions during the parameter estimation process. Hard constraints can be implemented using a Lagrange method or as penalty functions with an adjustable multiplier. By penalty function, we mean any interior or exterior penalty or barrier function or augmented Lagrange term that suitably imposes a constraint. By Lagrange term, we mean a Lagrange method or generalized Lagrange method that imposes a constraint and that satisfies the Kuhn Tucker conditions. This can be, for example, a method that employs a Lagrange multiplier for an active constraint or a method that employs a Kuhn Tucker multiplier under a complementarity condition (Fletcher, 1987).

With this background, henceforth we can address the long felt need for a method and system which substantially overcome or reduce the above noted problems in MRTA and its derivatives like CBMT, and in particular, for the imposition of relevant and useful information in the form of constraints on the model parameters while fitting the model to the data. Therefore, it is also desirable to provide a method and system for determining bounds on (a) the model parameters, (b) collections of model parameters, and (c) functions of model parameters, from a small set of individuals spanning a range of body types and sizes, and with the ability to improve and refine the bounds over time.

### SUMMARY

It is an objective of the invention to provide a method and system which substantially overcome or reduce the above noted problems in MRTA and its derivatives like CBMT, and in particular, for the imposition of relevant and useful information in the form of constraints on the model parameters while fitting the model to the data.

Another objective of the invention is to provide a method and system for determining bounds on the model parameters, collections of model parameters, and functions of model parameters from a small set of individuals spanning a range of body types and sizes, and with the ability to improve and refine the bounds over time.

Another objective of the invention is to provide a method and system for improving the numerical stability of fitting models to the collected data to provide an overall improvement in the quality of the model parameter estimates that are fit to the collected data.

In one embodiment, the system comprises a test probe unit having a mounting system with supports and restraints for the body part (for example, for a forearm, the system would have supports for the wrist and the elbow and a grip for the upper arm), a vibrator unit having a vibrator motor and a shaker motor shaft, a test probe with one or more sensors, and a means for positioning the test probe on the body part. In one embodiment, the test probe unit further comprises a controller having a processor and a memory containing computer readable and executable instructions which, when executed by the processor enables the test probe to provide functionality of receiving a signal from one or more sensors. In one embodiment, the controller is configured to perform multiple functions.

In one embodiment of the present invention, the subject or patient places his or her body part (the forearm in one or more embodiments) on the system mounts. The operator then positions and aligns the body part and then applies restraints for holding the body part in a preferred position. The operator then aligns a test probe to the initial test site and excites the vibration motor with an “excitation signal” while recording force and accelerance data. Further, prior to the excitation, the operator could apply a static force (a “static load”) within the range of static forces specified for the MRTA or CBMT that persists for the remainder of the testing procedure to reduce the amount of fluid under the probe between the surface of the skin and the bone. In another embodiment, the operator or test subject is fitted with a compression sleeve around the body part to assist in moving fluid away from the measurement area. In another embodiment, the compression sleeve is fitted prior to placement of the body part into the restraint system. In another embodiment, the compression sleeve is integrated into the test probe unit. After applying the static load and allowing adequate time for fluid under the probe to migrate away, for example, 5 seconds, a first excitation signal is also applied. In one embodiment, the first excitation signal lasts for at least 1, 2, 3, 4, 5, 6, 7, 8, 9, 10 or more seconds. In another embodiment, the first excitation signal lasts for at most 1, 2, 3, 4, 5, 6, 7, 8, 9, 10 or fewer seconds. In another embodiment, the first excitation signal induces transients in the mechanical frame and related structures, and the transients die out in approximately 5 seconds from the start of the first excitation signal. In one or more embodiments, multiple excitation signals (at least two) are applied sequentially, and the excitation signal(s) comprising the first 1, 2, 3, 4, 5, 6, 7, 8, 9, 10 or more seconds are trimmed from the data before analysis.

In one embodiment, a linearly swept (or chirp) transmit signal is used. By linearly sweeping over a range of frequencies in a fixed amount of time, a relationship between time and frequency is implicitly established. In one or more embodiments, the excitation signal is a “chirp”, also called a “swept chirp” or a “swept signal”, with amplitudes and angular velocities for each frequency adjusted to provide (a) an adequate signal to noise ratio over the frequencies of interest for both force and acceleration measurements, and (b) an overall smooth waveform to avoid exciting resonant frequencies of the mechanical frame and related structures. In another embodiment, the transducer generates one or more of a white noise test signal, a pink noise test signal, a swept frequency test signal, a pseudorandom noise, a periodic chirp, a sinusoidal signal, and a step function or other standard waveforms known to one of ordinary skill in the art.

In one embodiment, the chirp amplitude envelop begins with a lowest amplitude at a lowest frequency, follows a smoothly sigmoidal shape to its peak amplitude at the highest frequency, then symmetrically decrescendo's back to the lowest amplitude at the lowest frequency. In another embodiment, the rate of change of the chirp's angular velocity changes more slowly as the frequency increases, so that maximum dwell time occurs at the highest frequency, thereby injecting more power at those frequencies where dispersion is greatest (i.e., at the higher frequencies). In one or more embodiments, both chirp amplitude and rate of change of the chirp's angular velocity are combined to provide adequate, level, signal to noise ratios in all frequency bands of interest. In one embodiment, the chirp is at least 1, 2, 3, 4, 5, 6, 7, 8, 9, 10 or more seconds long. In another embodiment, the chirp is less than 5 seconds long, but may be repeated to create a stimulus waveform with at least 5 seconds duration. In one embodiment, the chirp spans the frequencies from 60 Hz to 1,200 Hz, while in another embodiment, it spans the frequencies from 50 Hz to 1,400 Hz, in another embodiment, it spans the frequencies from 40 Hz to 1,600 Hz, and in another embodiment, it spans the frequencies from 40 Hz to 1,200 Hz. In one or more embodiments, the chirp spans a frequency range having a minimum frequency of about 40, 45, 50, 55, 60, 65, 70, 75, 80 Hz or more and a maximum frequency of approximately 1600, 1500, 1400, 1300, 1200, 1100, 1000, 900, 800 Hz or less.

In one or more embodiments, the waveform for the chirp is tuned to the specific mechanical system being used (i.e., the mechanical frame, shaker motor, shaker motor mounting, patient restraints, etc.) and for a typical patient and body part (such as a forearm or lower leg) to reasonably level the magnitude spectrums of the force and acceleration data, to maintain an adequate signal to noise ratio in both force and acceleration measurements in all frequency bands of interest, and to avoid, as much as possible, excitation of resonances within the mechanical system.

In yet other embodiments, the primary excitation signal is derived from the swept chirp but is presented as a random signal with the same frequency power spectral density as the chirp. In one embodiment, the excitation signal is a uniform white noise sequence that has been transformed by well-known means to match the chirp's power spectral density. In another embodiment, the excitation stimulus is a Gaussian white noise sequence that has been transformed by well-known means to match the chirp's power spectral density. Those skilled in the art will realize that many types of noise distributions can be transformed into a random sequence that matches the power spectral density of the chirp, and thereby minimize excitation of spurious modes of vibration from the motor, the frame, the patient, and the combination of these.

In one or more embodiments, a small dither signal is applied throughout testing, beginning with the static load. The small dither signal keeps the vibrator motor active throughout the testing period so that the vibrator motor does not produce startup transients when the first, and subsequent, chirp or noise signals within the excitation signal begins, thereby eliminating the need for a second, third or additional sequential chirp or noise signal within the excitation signal, and thereby reducing testing time.

In another embodiment, the excitation signal is repeated with increasing amplitude to find the preferred excitation amplitude for the individual being tested. For example, the patient could be tested beginning with a low amplitude, then the test is repeated with a medium amplitude, and then it is repeated with a high amplitude, with all amplitudes staying within the range specified for MRTA or CBMT. Generally, higher excitation signal amplitudes work best for higher BMI and muscular individuals, but they cause excitation of lateral and torsional vibratory modes in low and moderate BMI/muscular individuals, which negatively impacts the parameter estimates of the primary bending modes for these smaller individuals. Therefore, the smallest excitation signal amplitude is desired that provides adequate signal to noise ratios. In one embodiment, the preferred excitation signal amplitude is determined by assessing the resulting data for the presence or absence of lateral and torsional vibratory modes and adequate signal to noise ratios over the frequencies of interest, and only the results for that preferred excitation signal amplitude are reported.

In another embodiment, testing is repeated with increasing static load force (e.g., small, medium, and high) within the range specified for MRTA or CBMT, to find the preferred static load force for the individual being tested. Generally, signal to noise ratios improve with higher static load for higher BMI and/or muscular individuals but lead to distortions in the modes of vibration for lower BMI and/or muscular individuals. Therefore, the smallest static load force is desired that provides adequate signal to noise ratios and minimizes distortion. In one or more embodiments, the preferred static load force is determined by assessing the resulting data for the presence or absence of distortions in the modes of vibration and adequate signal to noise ratios over the frequencies of interest, and only the results for that static load force are reported. In one or more embodiments, the static load force varies between about 1 N and about 30 N. In another embodiment, the static load force varies between about 3 N and about 30 N. In another embodiment, the static load force varies between about 3 N and about 25N. In other embodiment, the range of static loads is at least 3, 4, 5, 6, 7, 8, 9 10 N or more. In other embodiment, the range of static loads is at most 30, 25, 20 N or less.

In one or more embodiments, a crossed design of multiple static load forces and excitation signal amplitudes are applied sequentially, for example, starting with the low static load force, for which the low, medium, and high excitation signal amplitudes are applied sequentially, after which the static load force is increased to the medium static load force, and again the low, medium, and high excitation signal amplitudes are applied sequentially, after which the high static load force is applied, and the low, medium, and high excitation signal amplitudes are applied a final time. The preference for cycling the excitation signal amplitude while only increasing the static load forces is to minimize nonlinear creep and relaxation effects associated with fluid ingress back into the soft tissues under the probe that would occur if the static load were reduced. In one or more embodiments, the preferred static load force and excitation signal amplitude are determined by assessing the resulting data for the presence or absence of lateral and torsional vibratory modes and distortions, and adequate signal to noise ratios over the frequencies of interest, and only the results for that preferred static load force and excitation signal amplitude are reported. Another embodiment measures the differences in bone and soft tissue properties when static load force is later reduced.

In one or more embodiments, a single preferred static load force and excitation signal amplitude are applied during testing as determined by the individual's BMI and muscularity, thereby minimizing the time needed for testing the individual. The static load and signal amplitude are selected from a look-up table compiled from prior population data spanning a range of BMIs and muscularities over a range of static load forces and excitation amplitudes. In another embodiment, the single preferred static load force and excitation signal amplitude are selected based on a mathematical function derived from the prior population data. In another embodiment, the individual is tested multiple times, with the static load force and excitation signal amplitude being iteratively adjusted using a steepest descent method within the allowable ranges for MRTA and CBMT, by assessing the resulting data for the presence or absence of lateral and torsional vibratory modes and distortions, and for adequate signal to noise ratios over the frequencies of interest. In another embodiment, a bisection method is used in which the static load force and excitation signal amplitude are increased if the signal to noise ratios is below an acceptable threshold and decreased if lateral or torsional vibratory modes or distortions are detected, until a balance is achieved or until a maximum number of tests have been performed.

In one or more embodiments, the Levenberg-Marquardt optimizer/solver is applied to a cost function comprised of the sum of the weighted sum of squared frequency domain residuals and one or more of (a) the weighted sum of quadratic penalty functions, and (b) the sum of Lagrange terms. Those skilled in the art will realize that other optimizer/solvers can be applied to produce substantially similar results. The residuals are defined as the difference between the model's predicted frequency response function H_{pred}(jw) and the observed frequency response function H_{obs}(jw). Furthermore, since the residuals are complex-valued, for numerical efficiency, in one embodiment, they are broken into a purely real and a purely imaginary vector and the vectors are concatenated to form one real-valued, single-dimensional, residuals vector. Those skilled in the art will realize that other approaches can be used to handle complex valued residuals to produce substantially similar results. In one embodiment, the weights for the sum of squared frequency domain residuals are set to a value ranging from 0 to 1, such that the weights sum to 1, and span the frequencies from 40 Hz to 1600 Hz. In one preferred embodiment, the weights for the sum of squared residuals are set to a value of 0 for frequencies below 80 Hz, then to linearly increasing values from 0 to 1 between 80 Hz and 85 Hz, then to a value of 1 for frequencies between 85 Hz and 400 Hz, then to linearly decreasing values from 1 to 0.25 for frequencies between 400 Hz and 450 Hz, then to a value of 0.25 for frequencies between 450 Hz and 1200 Hz, then to a value of 0.125 for frequencies between 1200 Hz and 1400 Hz, and to a value of 0 above 1400 Hz. In another embodiment, the frame and shaker motor/assembly resonances are determined for the given design using methods known to those skilled in the art, and the weights for the sum of squared frequency domain residuals corresponding to those resonance frequencies are set to 0. In another embodiment, the Levenberg-Marquardt optimizer/solver is applied to the inverse frequency response function. In another embodiment, the Levenberg-Marquardt optimizer/solver is applied to a cost function comprised of the sum of the weighted sum of squared time domain residuals and one or more of (a) the weighted sum of penalty functions, and (b) the sum of Lagrange terms. In another embodiment, the penalty function is a quadratic penalty function. In another embodiment, the penalty function is a hyperbolic function. In another embodiment, the penalty function is sigmoidal in shape. Those skilled in the art will recognize that there are other forms of penalty functions that will achieve substantially similar results.

In one embodiment, the residuals are defined as the difference between the model's predicted time domain output force f_{pred}(t) and the observed time domain output force data f_{obs}(t), where the input to the model is the time domain displacement data x_{obs}(t) calculated by twice integrating the observed time domain accelerance data. In a related embodiment, the residuals are modified by filtering with a filter designed to implement the frequency domain weighting specified above for the frequency domain residuals. In another embodiment, the inverse model is used, so that the residuals are defined as the difference between the model's predicted time domain output displacement x_{pred}(t) and the observed time domain output displacement data x_{obs}(t) (calculated by twice integrating the observed time domain accelerance data), where the input to the model is the observed time domain force data f_{obs}(t). In a related embodiment, the residuals are modified by filtering with a filter designed to implement the frequency domain weighting specified above for the frequency domain residuals.

In one embodiment, a method for non-invasive measurement of mechanical properties of a cortical bone of an individual is disclosed. At one step, the patient or test subject positions a limb containing the bone of interest on the mounting system, for example, a left forearm so as to measure the properties of the left ulna. In another step, the operator aligns the body part and applies restraints to hold the body part in alignment, and then places a test probe unit against the individual's skin in the region of the bone for determining the mechanical properties. At another step, the test probe unit applies a static load force within the range specified for the MRTA or CBMT combined with a small dither signal, both of which continue throughout the measurement. At another step, after an adequate delay within the range specified for MRTA or CBMT to allow fluid between the probe and the bone to flow out from under the probe, the test probe unit is excited with a first chirp of five seconds duration while recording force f(t) and acceleration a(t). In an embodiment without the dither signal, after the first chirp, the test probe unit is excited with a second and third or more swept chirps and the first chirp is trimmed from the data record before analysis. At another step, Fourier transforms and power spectral densities are used to calculate an observed impedance curve H(jw). At another step, a Levenberg-Marquardt optimizer/solver is applied to a cost function comprised of the sum of a weighted sum of squared frequency domain residuals, a weighted sum of penalty functions, and a sum of Lagrange terms. In one embodiment, the weights for the sum of squared frequency domain residuals are set to a value ranging from 0 to 1, such that the weights sum to 1, and span the frequencies from 40 Hz to 1600 Hz.

Within the optimizer/solver, multiple internal steps iteratively refine the model parameter subject to the constraints until they converge or reach a terminal internal step. One of the internal steps updates an estimate of the model parameters. Based on that update, in another internal step, the estimated model parameters are converted to other model forms (PZ, TF, or BM) if needed to calculate the cost function, and the cost function is calculated, including the residuals between the model's predicted impedance curve and the observed impedance curve, the penalty functions, and the Lagrange terms. Another internal step determines if convergence or another termination criterion has been reached. If not, then another iteration occurs. Otherwise, the optimizer/solver step terminates and returns the final values of the estimated model parameters. At another step, EI and related output variables are calculated and displayed. At another step, key measurements, model parameters, and output variables are saved to a storage media.

In one embodiment, the fitted model is the PZ model, and poles and zeros are fit to the impedance curve H(jw). In this embodiment, the final estimates for the PZ model are converted to TF model form and BM model form to calculate EI and other related output parameters or variables for their display and storage. Another embodiment fits the TF model parameters to the impedance curve H(jw) and the final estimates are converted to the BM model form to calculate EI and other related output parameters or variables for their display and storage. The preferred embodiment fits BM model parameters to the impedance curve H(jw) and the final estimates are used to calculate EI and related output parameters or variables for their display and storage.

In summary, in the original embodiments of MRTA, force and accelerance data are collected in response to an excitation signal applied to a body part containing a bone of interest, the frequency response function is calculated, a dynamic system analyzer fits a PZ model (4 zeros and 2 poles) to the frequency response function, the values of the zeros and the poles are transferred to a computer, the computer algebraically converts the PZ model into TF model form and then again to the BM model form to arrive at an estimate of EI and other display parameters. In another MRTA embodiment, the TF model form is directly fitted to the frequency response function by the computer, and this approach seems to be the preferred embodiment in MRTA and subsequent derivatives like Bowman's CBMT. These earlier embodiments suffered from a variety of problems resulting in frequent errors and outright failure, creating a major barrier to successful commercialization. The new approach described in this disclosure remedies these long-standing problems by applying constraints to the model fitting step and also extends the embodiment options to include directly fitting the BM model parameters as the preferred approach. Below, further disclosures describe the formulation of the constraints as well as methods for determining bounds on the constraints and methods for numerically conditioning the data and the model parameters.

In one embodiment, the present invention provides for a method for non-invasive measurement of mechanical properties of a cortical bone of an individual, comprising:
positioning the body part of the individual containing the cortical bone of interest on a mounting system and applying restraints; and
placing a test probe unit against the region of the bone for determining the mechanical properties, wherein the test probe unit comprises one or more test probes, one or more vibrator units, one or more sensors, and a controller having one or more processors and a memory containing computer readable and executable instructions which, when executed by a processor, allows the controller to perform multiple functions comprising:
   a) applying a static load to a test site;
   b) exciting the test probe unit with a stimulus while recording force f(t) and acceleration a(t);
   c) processing the collected force and acceleration data;
   d) fitting a model to the processed data by applying an optimizer/solver that imposes one or more constraints on the estimated model parameters; and
   e) calculating EI, related output variables, or both.

In another embodiment, a dither signal is added to the static load to keep the vibration motor active throughout testing so that startup transients are not produced when the excitation stimulus begins.

In another embodiment, the excitation stimulus is comprised of one or more swept sinusoids (“chirps”) with a frequency amplitude profile matched to the equipment to minimize spurious vibrations and to produce an adequate signal to noise ratio for the force and acceleration data.

In another embodiment, the excitation stimulus is a uniform white noise signal with a frequency amplitude profile matched to the equipment to minimize spurious vibrations and to produce an adequate signal to noise ratio for the force and acceleration data.

In another embodiment, the excitation stimulus is a Gaussian white noise signal with a frequency amplitude profile matched to the equipment to minimize spurious vibrations and to produce an adequate signal to noise ratio for the force and acceleration data.

In another embodiment, the processed data are data that have been trimmed to remove unwanted transients and are then conditioned to be approximately zero mean and unit standard deviation.

In another embodiment, the processed data are further processed to calculate an impedance function H(jw), which can be the complex stiffness frequency response function or the complex compliance frequency response function.

In another embodiment, the optimizer/solver minimizes a cost function comprised of (a) a weighted sum of squared residuals or a weighted sum of absolute values of residuals, and (b) one or more of (i) a weighted sum of one or more penalty functions and (ii) one or more Lagrange terms.

In another embodiment, the optimizer/solver is an optimizer/solver capable of minimizing or maximizing nonlinear cost functions subject to linear and nonlinear constraints. In one embodiment, constraints are only linear, and the optimizer/solver is a quadratic program optimizer/solver. In other embodiments, the constraints are a mix of linear and nonlinear constraints, and for those, the optimizer/solver is a generalized nonlinear optimizer/solver. In another embodiment, the optimizer/solver is selected from the group consisting of a Nelder-Mead simplex optimizer/solver, a conjugant gradients optimizer/solver, a Gauss-Newton optimizer/solver, a Broyden-Fletcher-Goldfarb-Shanno (BFGS) optimizer/solver, a Davidon-Fletcher-Powell (DFP) optimizer/solver, a steepest descent optimizer/solver, a bisection optimizer/solver, and a Levenberg-Marquardt optimizer/solver. In another embodiment, the optimizer/solver is the Levenberg-Marquardt optimizer/solver. Those skilled in the art will realize that other optimizers/solvers can be used to produce substantially similar results.

In another embodiment, the residuals are defined as the difference between the model's predicted frequency response function H_{pred}(jw) and the observed frequency response function H_{obs}(jw). In another embodiment, the residuals are defined as the difference between the model's predicted time domain output force f_{pred}(t) and the observed time domain output force data f_{obs}(t), where the input to the model is the time domain displacement data x_{obs}(t) calculated by twice integrating the observed time domain accelerance data. In another embodiment, the residuals are defined as the difference between the model's predicted time domain output displacement x_{pred}(t) and the observed time domain output displacement data x_{obs}(t) calculated by twice integrating the observed time domain accelerance data, where the input to the model is the time domain force data f_{obs}(t).

In another embodiment, the model is a BM model. In another embodiment, the BM model is a 6, 7, 8, 9, or 12 Parameter BM Model or a variant of the 6, 7, 8, 9, or 12 Parameter BM Model. In another embodiment, the BM model is a finite element model of the body part that has been parameterized to 1, 2, 3, 4, 5, 6, 7, 8, 9, 10, or more parameters. The finite element method (FEM) works by breaking down a large problem into a handful of smaller problems called “finite elements.” The mathematical equations behind FEM are applied to create a simulation, or what's known as a finite element analysis (FEA).

In another embodiment, the imposed constraints include at least one constraint selected from the group consisting of: minimum and maximum system gain, system stability, minimum and maximum damping/settling time, non-negativity of physical parameters, minimum and maximum BM parameter values, minimum and maximum peak frequencies, relative peak frequencies, minimum and maximum flexural rigidity, non-negativity of the quadratic radicals in Kₛ, and non-vanishing denominator in the quadratic formula for Kₛ.

In another embodiment, a limited number of subjects spanning the range of BMIs and muscularities are tested to determine approximate population norms for defining constraint bounds.

In another embodiment, the subject is measured repeatedly at different points along a transit line in the transverse plane, and the results from the “sweet spot” (where the spurious modes of vibration are eliminated or minimized) are displayed and stored.

In another embodiment, the subject is measured repeatedly at different points, such as along a transit line in the transverse plane, along a line in the longitudinal direction within the sagittal plane, at different angles of the test probe relative to the cortical bone of interest, or combinations of transit lines, longitudinal lines, and angles, and the results from the “sweet spot” (where the spurious modes of vibration are eliminated or minimized) are displayed and stored.

### BRIEF DESCRIPTION OF THE DRAWINGS

FIG. 1 illustrates a perspective view of a system used for estimating in vivo the mechanical properties of a bone, for example, the left ulna of an individual, according to an embodiment of the present invention.

FIG. 2 illustrates a biomechanical (BM) model used in analyzing mechanical properties of a bone (for example, a left ulna), its overlying skin and soft tissue, and soft tissue in its surrounding peripheral region according to one embodiment of the present invention.

FIG. 3 illustrates a flowchart of a method for non-invasive measurement of mechanical properties of a cortical bone of an individual according to one embodiment of the present invention.

FIG. 4 illustrates a definition of the planes and axes relative to the bone, for example, left ulna being tested according to one embodiment of the present invention.

FIG. 5 illustrates a transverse cross section of the bone region, for example, left ulna of the individual, and stylized CBMT frequency response functions Hᵢ(jw) for discrete transit test points i across the skin over the ulna in the transverse plane according to one embodiment of the present invention.

FIG. 6 illustrates a flowchart of a method for performing constrained estimation of model parameters according to one embodiment of the present invention.

### DETAILED DESCRIPTION OF EXAMPLE EMBODIMENTS

Example embodiments of the disclosure now will be described more fully hereinafter with reference to the accompanying drawings, in which example embodiments are shown. The concepts discussed herein may, however, be embodied in many different forms and should not be construed as limited to the example embodiments set forth herein; rather, these embodiments are provided so that this disclosure will be thorough and complete, and will fully convey the scope to those of ordinary skill in the art. Like numbers refer to like elements but not necessarily the same or identical elements throughout.

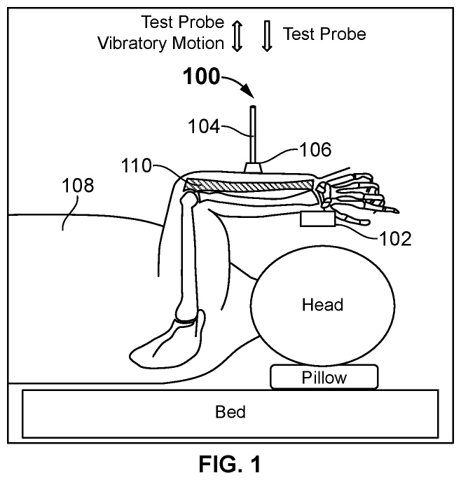

Referring to FIG. 1, a system 100 used for in vivo estimating the mechanical properties of a bone 110, for example, left ulna of an individual 108, according to an embodiment, is disclosed. In one embodiment, the system 100 is used for non-invasive measurement of mechanical properties of a cortical bone of an individual. In one embodiment, the system 100 substantially overcomes or reduces the problems in MRTA and its derivatives like CBMT by optimally imposing relevant and useful constraints on the model parameters while fitting the model to the data. In one embodiment, the system 100 is used for determining bounds on the constraints, including bounds on the model parameters, on collections of model parameters, and on functions of model parameters, from a small set of individuals spanning a range of body types and sizes, and with the ability to improve and refine the bounds over time. In one embodiment, the system 100 comprises a test probe unit having a mounting and restraining system, a wrist support 102, an elbow support, and an upper arm grip, and a vibrator unit 104 having a vibrator motor, a shaker motor shaft, and a test probe 106 with one or more sensors. In one embodiment, the test probe unit further comprises a controller having a processor and a memory containing computer readable and executable instructions which, when executed by the processor, is configured to perform a single function or multiple functions.

In one embodiment, the test subject or patient places his or her body part (the forearm in the preferred embodiment) on the mounting system of the system 100. The test probe 106 is placed against the region of the bone for determining the mechanical properties as described earlier.

In one embodiment, it is important to condition the sensor data (Force and Displacement in this particular embodiment, though it is applied to Force and Velocity in another embodiment, and to Force and Acceleration in a third embodiment) to maximize computational numerical precision for the model fit and the termination criteria. In another embodiment, each signal is transformed to zero mean and unit standard deviation.

In one or more embodiments, it is also important to condition the model parameters that are being estimated by the optimizer/solver. For the 7-Parameter BM model, each of the 7 parameters is transformed to have approximately zero mean and unit standard deviation to maximize computational numerical precision for the model fit and the termination criteria. Preferably, the transformation is based on population norms, which can be gathered over time as human data is collected. The transformations can also be a function of age, body type, and muscularity. In the current preferred embodiment, the parameter constraint bounds are used to establish the transformation, in which the midpoint of the range is defined as the parameter mean, and the range for each bound is defined as 1, 2, and 3 standard deviations in various embodiments for the purposes of transforming the parameters to zero mean and unit standard deviation. Those skilled in the art will realize that other transformations can be employed with similar utility, for example, a log-normal distribution, a beta distribution, etc. The specific distribution and its transformation parameters can be determined once population norms are established over a statistically significant number of individuals based on analysis of the histograms for each parameter. In one or more embodiments, once the data and parameters are transformed to an approximate zero mean and unit standard deviation as described above using the estimated mean and standard deviation of the data, and using the constraint bounds of the individual parameters, numerical stability seemed to be robust and insensitive to further refinement of the transformation parameters.

Referring to FIG. 2, a biomechanical (BM) model 200 used in analyzing mechanical properties of the bone, for example, left ulna and the overlying soft tissue region in one embodiment is disclosed. In one embodiment, the BM model 200 includes 7 parameters and accounts for the mass, stiffness, and damping of the skin and bone as well as parallel damping of soft tissues. Specifically, the 7-parameter model accounts for mass of skin (Mₛ) 202, stiffness of skin (Kₛ) 204, damping of skin (βₛ) 206, mass of bone (participating mass) (M_{b}) 208, stiffness of bone (K_{b}) 210, damping of bone (β_{b}) 212, and damping attributed to peripheral tissues (βₚ) 214. Other BM models used in MRTA and its derivative technologies like CBMT add dynamics for the end supports, resulting 9- and 12-parameter BM models, among others. When a subject being tested has a low BMI, the 7-Parameter BM Model may be reduced to a 6-parameter BM model, among others. While the present disclosure is applicable to numerous parametric mathematical models, for purposes of conveniently describing certain embodiments thereof, reference will be made to the 7-parameter model. However, one of skill in the art will recognize that reference to the 7-parameter model is not intended to be limiting and that the methods and systems disclosed herein are applicable to alternate parametric mathematical models.

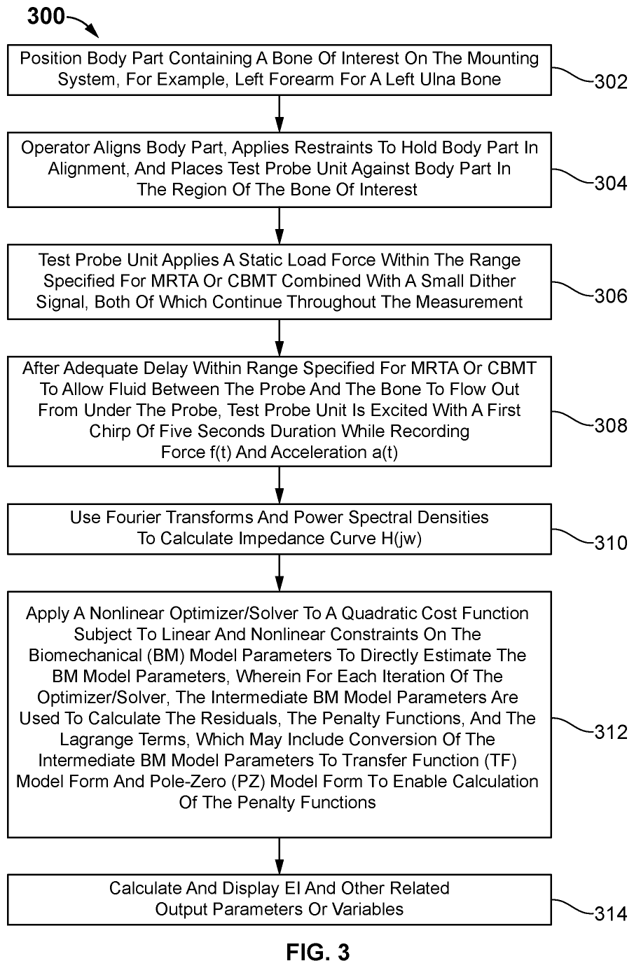

Referring to FIG. 3, a flowchart of a method 300 for non-invasive measurement of mechanical properties of a cortical bone of an individual is disclosed. Those skilled in the art will appreciate that present methods, systems, and apparatuses can employ both digital and analog equipment. One skilled in the art will appreciate that provided herein is a functional description and that the respective functions may be performed by software, hardware, or a combination of software and hardware. At step 302, the patient or test subject positions his or her body part on the mounting system, for example, the left forearm, so that the bone of interest may be measured, in this example, the left ulna bone. At step 304, the operator aligns the body part, applies restraints to hold the body part in a preferred position, and the test probe unit is placed against the region of the bone for determining the mechanical properties. At step 306, the test probe unit applies a static load force within the range of static load forces specified for the MRTA or CBMT combined with a small dither signal, both of which continue for the remainder of the measurement. In Step 308, after adequate time has elapsed for most of the fluid under the probe (between the surface of the skin and the bone) to migrate away, and while recording force f(t) and acceleration a(t), the probe is excited with a chirp of five seconds duration. At step 310, Fourier transforms and power spectral densities are used to calculate impedance curve H(jw). At step 312, the BM model parameters are directly estimated using, for example, a Levenberg-Marquardt optimizer/solver applied to a cost function comprised of the sum of (a) a weighted sum of squared frequency domain residuals, (b) a weighted sum of penalty functions, and (c) a sum of the Lagrange terms. The penalty functions and Lagrange terms impose the applicable constraints. In one or more embodiments, all constraints are implemented as penalty functions so there are no Lagrange terms. At step 314, EI and other related output parameters or variables are calculated, displayed, and saved to a storage media.

In another embodiment, at step 312, instead of fitting the BM model parameters, PZ model parameters are fit to the impedance curve H(jw) using the optimizer/solver, and at each iteration of the solver, the intermediate PZ model parameters are also converted to transfer function (TF) model form and to biomechanical (BM) model form to calculate the constraint costs (the penalty functions and/or the Lagrange terms). After convergence, in preparation for step 314, the PZ model parameters are also converted to TF and BM model forms to enable calculation of EI and other important output parameters or variables. In another embodiment, the TF model parameters are fit to the impedance curve H(jw) using the optimizer/solver, and at each iteration of the solver, the intermediate TF model parameters are also converted to PZ and BM model forms to calculate the constraint costs (the penalty functions and/or the Lagrange terms). After convergence, in preparation for step 314, the TF model parameters are also converted to PZ and BM model forms to enable calculation of EI and other important output variables. In one or more embodiments, wherein the BM model parameters are fit to the impedance curve H(jw) using the optimizer/solver, at each iteration of the solver, the intermediate BM model parameters are also converted to TF and PZ model forms to calculate the constraint costs (the penalty functions and/or the Lagrange terms). After convergence, in preparation for step 314, the BM model parameters are also converted to PZ and TF model forms to enable calculation of EI and other important output variables.

As described above, in some embodiments, the optimizer directly estimates the biomechanical model parameters, in others, it directly estimates the poles and zeros, and in others, it estimates the transfer function coefficients. In the embodiments, subject to the constraints, the optimizer fits the chosen model to the frequency response function H(jw), which describes the frequency domain behavior of the mechanical system in response to measured force and displacement (wherein displacement is usually obtained by twice integrating a measured acceleration). In other embodiments, subject to the constraints, the optimizer fits the chosen model to the time domain behavior of the mechanical system according to the measured force and displacement (wherein displacement is usually obtained by twice integrating a measured acceleration). In other embodiments, the optimizer fits the chosen model to the response of the measured force and velocity (wherein velocity is usually obtained by once integrating a measured acceleration), and in other embodiments, the optimizer fits the chosen model to the response of the measured force and the measured acceleration.

The following describes the embodiments in which the optimizer fits the chosen model to H(jw) utilizing the measured force and displacement wherein displacement is obtained by twice integrating a measured acceleration. Those skilled in the art will realize the embodiments utilizing directly measured displacement, directly measured velocity, and directly measured acceleration are developed similarly. Furthermore, H(jw) is a generalized impedance function that can be either the complex stiffness F(jw)/X(jw) or the complex compliance X(jw)/F(jw), where F(jw) is the Fourier transform of force f(t) and X(jw) is 1/(jw) 2 times the Fourier transform of acceleration a(t). In practice, H(jw) is often calculated using Fourier power spectral densities and its variants (see, for example, Vold et al., 1984). Complex stiffness will be used in the following description, and those skilled in the art will realize the embodiments utilizing complex compliance use the same arguments but with their reciprocal representations.

In the equation below for the complex stiffness (Eq. 1), the Pole-Zero (PZ) model form is parameterized by the poles p and zeroes z, and the Transfer Function (TF) model form is parameterized by the coefficients of s, which are the a's and b's in the equations below, where G is the gain of the system and s (equal to σ+jw) is the Laplace Transform variable. Both model forms are rational polynomials in s, and if the system is stable (or more precisely, the region of convergence of the Laplace Transform includes the jw axis), then jw can be substituted for s to obtain the Fourier Transform. These forms can be algebraically converted from one model form to the other.

Converting from PZ model form to TF model form requires simple polynomial multiplication, while converting from TF model form to PZ model form requires factoring (root finding) for the numerator and denominator polynomials.

$$\frac{F ( s )}{X ( s )} = G \frac{( s - {z}_{m} ) ( s - {z}_{m - 1} ) … ( s - {z}_{1} )}{( s - {p}_{n} ) ( s - {p}_{n - 1} ) … ( s - {p}_{1} )} = G \frac{{s}^{m} + {b}_{m - 1} {s}^{m - 1} + … + {b}_{1} s + {b}_{0}}{{s}^{n} + {a}_{n - 1} {s}^{n - 1} + … + {a}_{1} s + {a}_{0}} \qquad ( 1 )$$

Algebraically converting from BM model form to the TF model form is described herein for the 7-Parameter BM model. Those skilled in the art will realize that similar conversions can be made for the other BM models. Eq. 2 shows a fourth order TF model representing the Force-Displacement relationship, where G (the system gain) is equal to Mₛ, the mass of the skin, and the a's and b's are uniquely determined from the 7-Parameter BM model as shown in Eq. 3 through Eq. 8.

$$\frac{F ( s )}{X ( s )} = {M}_{s} \frac{{s}^{4} + {b}_{3} {s}^{3} + {b}_{2} {s}^{2} + {b}_{1} s + {b}_{0}}{{s}^{2} + {a}_{1} s + {a}_{0}} \qquad ( 2 )$$

$${b}_{0} = \frac{{K}_{s} {K}_{b}}{{M}_{s} {M}_{b}} \qquad ( 3 )$$

$${b}_{1} = \frac{{K}_{b} ( {β}_{s} + {β}_{p} ) + {K}_{s} ( {β}_{b} + {β}_{p} )}{{M}_{s} {M}_{b}} \qquad ( 4 )$$

$${b}_{2} = \frac{{K}_{s} + {K}_{b}}{{M}_{b}} + \frac{{K}_{s}}{{M}_{s}} + \frac{{β}_{s} ( {β}_{b} + {β}_{p} ) + {β}_{b} {β}_{p}}{{M}_{s} {M}_{b}} \qquad ( 5 )$$

$${b}_{3} = \frac{{β}_{s} + {β}_{b}}{{M}_{b}} + \frac{{β}_{s} + {β}_{p}}{{M}_{s}} \qquad ( 6 )$$

$${a}_{0} = \frac{{K}_{s} + {K}_{b}}{{M}_{b}} \qquad ( 7 )$$

$${a}_{1} = \frac{{β}_{s} + {β}_{b}}{{M}_{b}} \qquad ( 8 )$$

The conversion from the TF model form to 7-Parameter BM model form requires solving for the larger root of a quadratic equation in Kₛ (Eq. 11, using the definitions in Eq.'s 9 and 10). Once determined, the remaining parameters of the 7-Parameter BM model can be calculated from Eq. 12 through Eq. 16.

$${D}_{0} {=}^{△} Δ {M}_{s} ( {b}_{2} - {a}_{1} {b}_{3} + {a}_{1}^{2} - {a}_{0} ) \qquad ( 9 )$$

$${D}_{1} {=}^{△} Δ {M}_{s} [ {a}_{0} ( {b}_{3} - {a}_{1} ) - {b}_{1} ] \qquad ( 10 )$$

$${K}_{s} = larger root of \{ {K}_{s}^{2} ( 4 {a}_{0} - {a}_{1}^{2} ) - {K}_{s} ( 2 {a}_{1} {D}_{1} + 4 {a}_{0} {D}_{0} + 4 {M}_{s} {b}_{0} ) + 4 {M}_{s} {b}_{0} {D}_{0} - {D}_{1}^{2} = 0 \} \qquad ( 11 )$$

$${β}_{s} = \frac{2 {K}_{s} ( {K}_{s} - {D}_{0} )}{{a}_{1} {K}_{s} + {D}_{1}} \qquad ( 12 )$$

$${M}_{b} = \frac{4 {K}_{s}^{2} ( {K}_{s} - {D}_{0} )}{{( {a}_{1} {K}_{s} + {D}_{1} )}^{2}} \qquad ( 13 )$$

$${K}_{b} = \frac{{M}_{s} {b}_{0} {K}_{s}}{{a}_{0} {K}_{s} - {M}_{s} {b}_{0}} \qquad ( 14 )$$

$${β}_{b} = {M}_{b} {a}_{1} - {β}_{s} \qquad ( 15 )$$

$${β}_{p} = {M}_{s} ( {b}_{3} - {a}_{1} ) - {β}_{s} \qquad ( 16 )$$

Those skilled in the art will realize that the solution to an unconstrained quadratic equation as in Eq. 11 can present multiple problems. For example, (a) with finite precision arithmetic, the numerical solution for the roots can become ill conditioned if two numbers that are nearly identical must be differenced or if a very small number must be summed with a very large number, (b) the radical can become negative leading to BM model parameters that are complex valued, (c) the denominator can approach zero leading to a singularity and/or numerical instability, (d) the magnitude of the squared term in the radical can generate an overflow, etc.

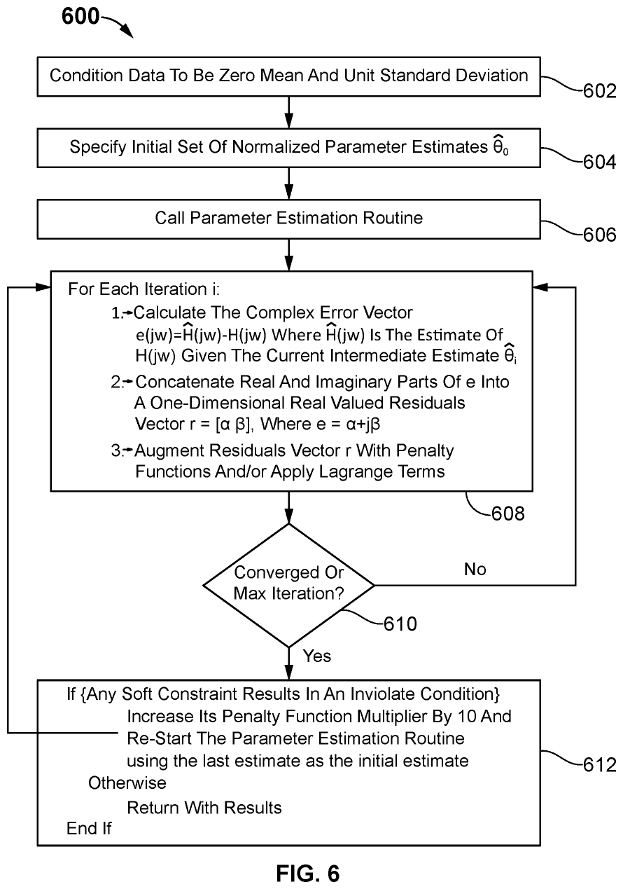

In addition, nothing in these equations prevents the BM model parameters from becoming negative. Any one of these problems can (and in practice often do) lead to nonsensical values for the BM model parameters. Similar problems can arise when finding the roots of the TF model's numerator and denominator polynomials when converting it to PZ model form. The embodiments described here avoid these problems by careful numerical conditioning and by imposing constraints that prevent nonsensical values. The constraints are disclosed following the detailed descriptions of FIG. 6.

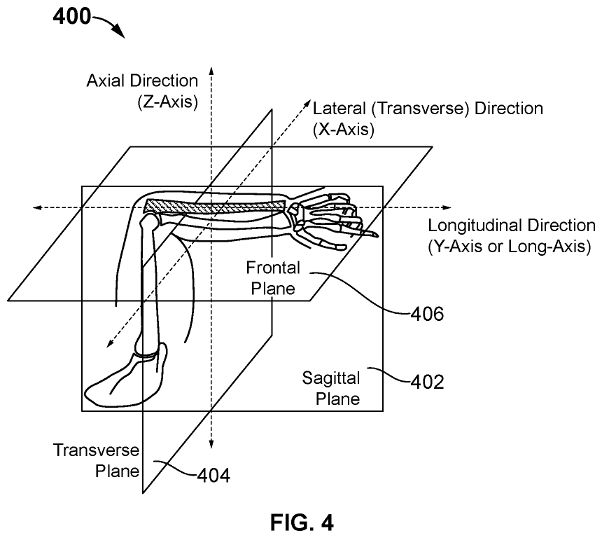

FIG. 4 illustrates the definitions (400) of the planes and axes relative to the bone, for example, a left ulna being tested in one of the disclosed embodiments. In this embodiment, the definitions include a sagittal plane 402, a transverse plane 404, and a frontal plane 406, and the X, Y, and Z axes and their directions.

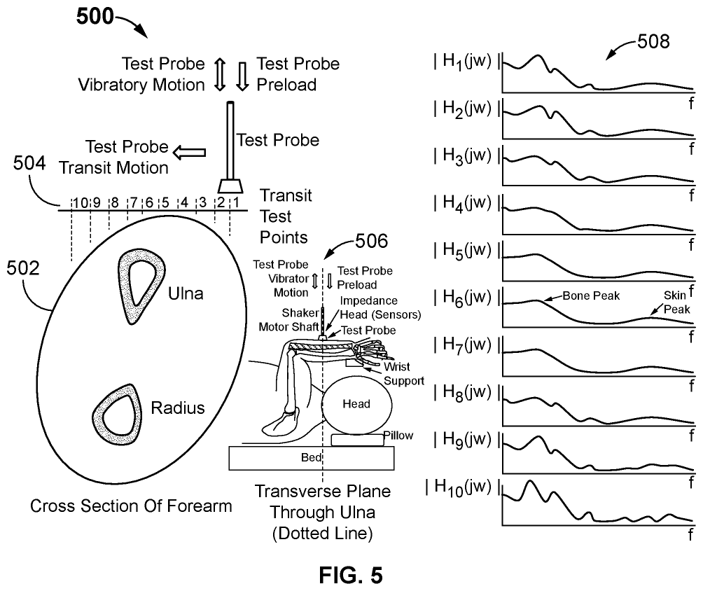

FIG. 5 illustrates CBMT (Bowman's Method) 500 applied to a cross section 502 of the bone region, for example, left ulna of the individual and MRTA measurements at discrete transit points (504) across the skin over the ulna in the transverse plane (506) is disclosed. In one embodiment, this is implemented using a robot to position the test probe unit and apply the test probe to the series of test sites. In another embodiment, this is implemented using a motorized gantry to position the test probe unit and apply the test probe to the series of test sites. In another embodiment, the test probe unit and test probe are positioned by the operator. In yet another embodiment, a mix of robot or motorized gantry with operator intervention is used to position the test probe unit and apply the test probe to the test sites. In these embodiments, MRTA measurements are repeated at discrete transit points across the skin over the ulna of the individual in the transverse plane. At least one test point in the transit (the “sweet spot”) should minimize lateral and/or torsional vibrations, as shown in the plots of |H(jw)| (508) for transit test points 5-7. In plots of |H(jw)| (508) for transit test points 1-4 and 8-10, additional modes of vibration are present, confounding signal analysis. Bowman's Method breaks down for individuals with smaller BMI as well as those with larger BMI because of the problems described above in this disclosure. The current invention solves these problems by applying numerical conditioning and parameter constraints during model fitting as described above and in the following text.

In FIG. 6, a flowchart 600 of an iterative method for imposing constraints during parameter estimation is disclosed. In one embodiment, the constraints are readily derived and imposed on direct estimates of the BM parameters but can also be applied to PZ and TF model parameters through Lagrange multiplier methods or penalty functions. To maximize numerical precision, it is generally considered best practice to: (a) condition the data (e.g., normalize by mean and variance) (602); (b) condition the model parameters (e.g., normalize by mean and variance) (604); (c) tune termination criteria (max. cost function, min. rate of decrease of cost function and/or flatness of the gradient, maximum number of iterations) to balance the goodness of fit (performance improvements) with maximum time for the fit/failure to converge; (d) concatenate the real and imaginary parts of the residuals into a one-dimensional vector (608); (e) append the residuals error vector to include penalty functions (608); (f) apply Lagrange terms that satisfy the Kuhn-Tucker conditions for hard constraints (if used) (608); (g) apply tests on the activation of the constraints and iterate to achieve desired “hardness” of the constraints (612). The constraints are imposed in step 608 by the penalty functions and Lagrange terms and are described in further detail below.

The specific details of constrained parameter estimation as shown in FIG. 6 are as follows. At step 602, the data is conditioned to be zero mean and unit standard deviation. At step 604, an initial set of normalized parameter estimates \hat{θ}₀ is specified. At step 606, the parameter estimation routine is called, which then iterates until convergence (608, 610, 612). At step 608, for each iteration i, several sub-steps are executed. First, the complex error vector e(jw)=Ĥ(jw)−H(jw) is calculated, where Ĥ(jw) is the estimate of H(jw) given the current intermediate estimate \hat{θ}i. Second, the real and imaginary parts of e are concatenated into a one-dimensional real valued residuals vector r=[αβ], where e=α+jβ. Third, the residuals vector r is augmented with penalty functions, and Lagrange terms are applied if needed. At step 610, termination conditions are checked, such as convergence of the parameter estimates or reaching the maximum number of iterations. If termination conditions have not been met, program execution is transferred back to step 608 for another iteration step. If termination conditions have been met, program execution is transferred to step 612, where, if any constraints imposed by penalty functions result in inviolate conditions, their respective penalty function multipliers are increased by a factor of 10 and program execution is transferred to step 608 to begin the iterative procedure again. Otherwise, step 612 returns with the model fit results.

In one or more embodiments, the BM model parameters are inherently forced to be real-valued without the need for imposing potentially disastrous and unrealistically strict rejection criteria as in Bowman's Method by instead estimating the BM model parameters directly using a non-linear curve fitting algorithm like the Levenberg-Marquardt algorithm. Furthermore, estimating the BM model parameters directly also readily enables the imposition of relevant and meaningful information based on the biophysics and on population norms, in the form of constraints on the BM model parameters. Therefore, in one or more embodiments, the Levenberg-Marquardt solver is used to fit the 7-Parameter BM model to the collected data. Those skilled in the art will realize that this same approach will work for other BM models besides the 7-Parameter model as well as for TF and PZ models with clever conversion of the constraints into these model spaces with utilization of convex hulls for those constraints that become non-convex. Those skilled in the art will also realize that, alternatively, repeated estimation with multiple initial guesses spanning the divergent non-convex constraint spaces can enable parameter estimation within the TF and PZ spaces. Those skilled in the art will further realize that constraints developed in the BM model space, when applied to direct estimation of the TF or PZ parameters, can be implemented perhaps most easily in the form of penalty function(s), that is, as functions of the BM model parameters that are themselves algebraically calculated from the estimated TF or PZ parameters. Those skilled in the art will further realize that other optimizers/solvers can also be used instead of the Levenberg-Marquardt optimizer/solver used in an embodiment. In one or more embodiments, the optimizer/solver is selected from the group consisting of a Nelder-Mead simplex optimizer/solver, a conjugant gradients optimizer/solver, a Gauss-Newton optimizer/solver, a Broyden-Fletcher-Goldfarb-Shanno (BFGS) optimizer/solver, a Davidon-Fletcher-Powell (DFP) optimizer/solver, a steepest descent optimizer/solver, a bisection optimizer/solver, a Levenberg-Marquardt optimizer/solver, and, when the constraints are only linear, a quadratic program solver.

Those skilled in the art will realize that, once the method and system are developed for use with BM models, the desire to work with the TF and PZ models diminishes. However, those skilled in the art will also realize that the context will determine whether the BM model space or the other model spaces are best for a given application, and so, in the following, example embodiments are described further for each model space.

In some embodiments, the constraints are converted from their source model space in which they were derived (BM, TF, and PZ model spaces), to the model space in which the parameters are to be estimated (BM, TF, or PZ model spaces), and convex hulls are created for any non-convex constraints.

In other embodiments, the constraints are converted from their source model space in which they were derived (BM, TF, and PZ model spaces), to the model space in which the parameters are to be estimated (BM, TF, or PZ model spaces), and if any are non-convex, the model fitting algorithm optimizer/solver is repeatedly called with initial parameter estimates made to span the model space created by the converted constraints, to find the global minimum instead of a local minimum within the non-convex space. The set of convergent parameters that fit the goodness of fit criteria best are then used as the final solution reported to the user/operator.

In yet other embodiments, the constraints are not converted from their source model space in which they were derived (BM, TF, and PZ model spaces) but are instead applied as penalty functions in the model space in which the parameters are to be estimated (BM, TF, or PZ model spaces). Some of these latter embodiments require the algebraic conversion of the estimated parameters into another model space to enable calculation of the penalty functions.

In one or more embodiments, the constraints are based on the biophysics and observed system behavior over a small number of individuals ranging over the various body-types of interest. In one embodiment, soft constraints on the BM model parameters are determined based on a small population of individuals, as described below, and are implemented as exterior quadratic penalty functions with parameterized multipliers that enable dynamic tuning of the penalties. In another such embodiment, the soft constraints on the BM Model parameters are implemented as interior penalty functions with steep growth in the penalties as the parameters approach the constraint boundaries from within the allowable constraint space. In these embodiments, if one or more soft constraints must remain inviolate, then if any are violated, those soft constraints are made increasingly hard by increasing their respective multipliers until the constraints are no longer violated, as described in step 612 of FIG. 6.

In another embodiment, the constraints that must remain inviolate (e.g., non-negativity) are imposed as hard constraints using Lagrange methods that satisfy the Kuhn-Tucker conditions. Those skilled in the art will realize that other methods may be employed that also satisfy and impose the Kuhn-Tucker conditions.

The following examples describe some of the commonly known (or readily known) forms of information that can be converted into constraints on the model parameters that are suitable for use with nonlinear optimizers. Those skilled in the art will realize that other information can be formulated as constraints on the parameters, that the following list is intended to be illustrative only.
(1) Minimum and maximum system gain G. The population-based range constraints on BM model parameters (see below) inherently impose minimum and maximum gains, but those do not account for combinations of parameters, and so the actual minimum and maximum system gains is a preferred embodiment in those models where the BM model's gain G is a combination of multiple parameters. For the 7-Paramater BM model, the system gain G is equal to the participating mass of the skin, and its range is imposed by the individual parameter constraints.
(2) System stability. Constraints on the system stability are inherently included in the minimum and maximum bounds on the parameters for the 7-Parameter BM model (see below). If other model spaces are used, explicit bounds for system stability may be imposed by using penalty functions applied to the poles and zeros of the PZ model.
(3) Minimum and maximum system damping/settling time. Minimum and maximum system damping/settling time likewise can be set though conditions on the locations of the poles and zeros of the PZ model, through penalty functions or Lagrange terms depending on the form of the model (PZ, TF, or BM) being fit.
(4) Non-negativity of physical parameters (masses, dashpots, spring constants). Some of these constraints are inherently included in the minimum and maximum BM model parameter bounds (see below) but are left intact in a preferred embodiment because the model parameter bounds are implemented as soft constraints, whereas the non-negativity of physical parameters are implemented as hard constraints. In another embodiment, the hard constraints for non-negativity are removed because, if any parameter drifts outside its lower bound, the multiplier for its lower bound is increased until that parameter no longer violates the non-negativity condition.
(5) Minimum and maximum BM model parameters. One or more embodiments impose these parameter extents as soft constraints. However, because there are slight differences in the various electro-mechanical designs for MRTA/CBMT in the literature, in the preferred embodiment, the parameter bounds should preferably be determined by collecting data for a specific electro-mechanical MRTA/CBMT design on a small number of individuals spanning the range of body types and disease states, for example, from 10 to 20 individuals as follows: (a) four to eight individuals with BMIs ranging from underweight (<18.5) to healthy (18.5 to <25.0) to overweight (25.0 to <30) to obese (≥30); (b) three to six individuals with muscularity ranging from minimal to high; and (c) three to six individuals with varying severity of osteoporosis. Only those data with little noise, and for CBMT, with a clear “sweet spot” that occurs for at least one of the CBMT transit points, should be used for establishing these constraints. These constraints should be updated and refined over time as more data are collected and statistically significant population norms are established as a function of age (including children), gender, height, weight, BMI, muscularity, disease state, and other relevant biometrics, by following an experiment design similar to the FDA guidelines for establishing population norms for sonometers using various testing arms and biometrics.

A preferred embodiment has been optimized over a small group of individuals as described above for adults with BMI between 18 and 25, and then extrapolated to the general population using supplemented data from the literature for the 7-Parameter BM model parameters for adult human ulna, and has resulted in the following bounds:

| | General Adult | | | |
|---|---|---|---|---|
| | Population | | Adults 18 ≤ BMI < 25 | |
| Parameter | Minimum | Maximum | Minimum | Maximum |
| M_{b} (kg) | 0.0080 | 0.1300 | 0.0159 | 0.0520 |
| K_{b} (N/m) | 8,000 | 500,000 | 55,000 | 135,000 |
| β_{b} (Ns/m) | 2.5 | 122.0 | 24.6 | 61.0 |
| Mₛ (kg) | 0.0057 | 0.5650 | 0.0113 | 0.0137 |
| Kₛ (N/m) | 16,000 | 2,000,000 | 150,000 | 400,000 |
| βₛ (Ns/m) | 0.20 | 28.00 | 2.00 | 14.00 |
| βₚ (Ns/m) | 1.00 | 73.00 | 9.93 | 36.60 |

(6) Minimum and maximum peak frequencies. In the frequency response function of the 7-Parameter BM model of the ulna, two peak frequencies are defined as the bone (F_{b,r}) and skin (F_{s,r}) peaks, or resonant frequencies, because they are attributed primarily to the bone or skin constituent components of the model (even though they are the result of combinations of various constituent model components). In some embodiments, F_{b,r_min} and F_{b,r_max} are respectively 100 Hz and 350 Hz, 75 Hz and 400 Hz, or 50 Hz and 450 Hz for human ulna. In some embodiments, F_{s,r_min} and F_{s,r_max} are respectively 450 Hz and 1200 Hz, 350 Hz and 1400 Hz, or 300 Hz and 1600 Hz for human ulna.
(7) Relative peak frequencies. Relationships between the two frequency peaks in the frequency response function of the 7-Parameter BM model of the ulna can provide useful constraints since the frequency of the bone peak is always less than the frequency of the skin peak. In one or more embodiments, the constraint is F_{s,r}>F_{b,r}+F_{offset}, where F_{offset} is 25 Hz, 100 Hz, 200 Hz, or more than 200 Hz. In another embodiment, F_{offset} is based on BMI and muscularity. In other preferred embodiments, the constraint is instead F_{s,r}>F_{b,r}×Fᵣₐₜᵢₒ where Fᵣₐₜᵢₒ is 1.1, 1.5, 2.0, 2.5, or more than 2.5 for human ulna. In another embodiment, Fᵣₐₜᵢₒ is based on BMI and muscularity.
(8) Minimum and maximum flexural rigidity (EIₘᵢₙ and EIₘₐₓ). In one or more embodiments, EIₘᵢₙ and EIₘₐₓ are respectively 8 and 125 Nm², 5 and 135 Nm², or 2 and 145 Nm² for human ulna. In another embodiment, constraints on EIₘᵢₙ and EIₘₐₓ are not imposed and so EI is allowed to range freely.
(9) Non-negativity of the quadratic radicals. In this constraint, the terms under the radical in the classical quadratic formula for Kₛ in Eq. 11 can be implemented as:

$${( 2 {a}_{1} {D}_{1} + 4 {a}_{0} {D}_{0} + 4 {M}_{s} {b}_{0} )}^{2} - 4 ( 4 {a}_{0} - {a}_{1}^{2} ) ( 4 {M}_{s} {b}_{0} {D}_{0} - {D}_{1}^{2} ) \geq 0 \qquad ( 17 )$$

However, embodiments with direct estimation of the BM model parameters, as in the preferred embodiment, avoid the need for this constraint because all BM model parameters are inherently real valued, and further, because they are estimated directly, the quadratic equation in Kₛ does not need to be solved.
(10) Non-vanishing denominator in the quadratic formula for Kₛ. Referring to Eq. 11, this becomes the constraint, |4a₀−a₁²|>ϵ, where ϵ is a small number that prevents numerical instability in the solution for the roots. However, embodiments with direct estimation of the BM model parameters, as in the preferred embodiment, avoid the need for this constraint because the BM model parameters are estimated directly so that the quadratic equation in Kₛ does not need to be solved.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described with reference to block diagrams and flowchart illustrations of methods, systems, apparatuses, and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The methods and systems can employ artificial intelligence (AI) techniques such as machine learning and iterative learning. Examples of such techniques include, but are not limited to, expert systems, case-based reasoning, Bayesian networks, behavior-based AI, neural networks, fuzzy systems, evolutionary computation (e.g., genetic algorithms), swarm intelligence (e.g., ant algorithms), and hybrid intelligent systems (e.g., Expert inference rules generated through a neural network or production rules from statistical learning).

Although the features, functions, components, and parts have been described herein in accordance with the teachings of the present disclosure, the scope of coverage of this patent is not limited thereto. On the contrary, this patent covers all embodiments of the teachings of the disclosure that fairly fall within the scope of permissible equivalents.

Many modifications and other implementations of the disclosure set forth herein will be apparent having the benefit of the teachings presented in the foregoing descriptions and the associated drawings. Therefore, it is to be understood that the disclosure is not to be limited to the specific implementations disclosed and that modifications and other implementations are intended to be included within the scope of the appended claims. Although specific terms are employed herein, they are used in a generic and descriptive sense only and not for purposes of limitation.

### REFERENCES

Steele C R, Zhou L J, Guido D, Marcus R, Heinrichs W L, Cheema C. Noninvasive Determination of Ulnar Stiffness from Mechanical Response—In Vivo Comparison of Stiffness and Bone Mineral Content in Humans. J Biomech Eng. 1988 May; 110 (2): 87-96. doi: 10.1115/1.3108423. PMID: 3379938.
Steele, Charles R. (1991). Bone/Tissue Analyzer and Method (U.S. Pat. No. 5,006,984). U.S. Patent and Trademark Office.
Bowman, L, Arnold, P A, Ellerbrock E R. (2019). Systems and Methods for Establishing the Stiffness of a Bone Using Mechanical Response Tissue Analysis (U.S. Pat. No. 10,299,719 B2). U.S. Patent and Trademark Office.
Bowman L, Loucks A B. Improvements to Mechanical Response Tissue Analysis. MethodsX. 2019 Oct. 14; 6:2408-2419. doi: 10.1016/j.mex.2019.10.004. PMID: 31687360; PMCID: PMC6820268.
Bowman L, Loucks A B. In Vivo Assessment of Cortical Bone Fragility. Curr Osteoporos Rep. 2020 February; 18 (1): 13-22. doi: 10.1007/s11914-020-00558-7. PMID: 32088857; PMCID: PMC7067717.
Bowman, L, Loucks, A B. (2021). Methods for Establishing the Stiffness of a Bone Using Mechanical Response Tissue Analysis (US Patent Application Publication No. US 2021/0045636 A1). U.S. Patent and Trademark Office.
Fletcher, R. Practical Methods of Optimization, 2^{nd} Ed. 1987. John Wiley & Sons Ltd. ISBN 0471915475. doi: 10.1002/9781118723203
H. Vold, J. Crowley, and G. T. Rocklin. “New Ways of Estimating Frequency Response Functions.” Sound and Vibration. Vol. 18, Iss. 11, 1984, pp. 34-38.

What is claimed is:

1. A method for non-invasive measurement of mechanical properties of a cortical bone of an individual, comprising:
positioning the body part of the individual containing the cortical bone of interest on a mounting system and applying restraints; and
placing a test probe unit against the region of the bone for determining the mechanical properties, wherein the test probe unit comprises one or more test probes, one or more vibrator units, one or more sensors, and a controller having one or more processors and a memory containing computer readable and executable instructions which, when executed by a processor, allows the controller to perform multiple functions comprising:
f) applying a static load to a test site;
g) exciting the test probe unit with a stimulus while recording force f(t) and acceleration a(t);
h) processing the collected force and acceleration data;
i) fitting a model to the processed data by applying an optimizer/solver that imposes one or more constraints on the estimated model parameters; and
j) calculating EI, related output variables, or both.

2. The method of claim 1, wherein a dither signal is added to the static load to keep the vibration motor active throughout testing such that startup transients are avoided or minimized when the excitation stimulus begins.

3. The method of claim 1, wherein the excitation stimulus is comprised of one or more swept sinusoids (“chirps”) with a frequency amplitude profile matched to the equipment to minimize spurious vibrations and to produce an adequate signal to noise ratio for the force and acceleration data.

4. The method of claim 1, wherein the excitation stimulus is a white, pink, or other noise signal that is uniform, Gaussian, or other shape, with a frequency amplitude profile matched to the equipment to minimize spurious vibrations and to produce an adequate signal to noise ratio for the force and acceleration data.

5. The method of claim 1, wherein the processed data are data that have been trimmed to remove unwanted transients and wherein the processed data are conditioned to be approximately zero mean and unit standard deviation.

6. The method of claim 1, wherein the processed data are further processed to calculate an observed impedance function H_{obs}(jw), which can be the complex stiffness frequency response function or the complex compliance frequency response function, which is then normalized to be approximately zero mean and unit standard deviation.

7. The method of claim 1, wherein the optimizer/solver minimizes a cost function comprised of (a) a weighted sum of squared residuals or a weighted sum of absolute values of residuals, and (b) a weighted sum of (i) one or more penalty functions, (ii) one or more Lagrange terms, or (iii) both one or more penalty functions and one or more Lagrange terms.

8. The method of claim 7, wherein the optimizer/solver is an optimizer/solver capable of minimizing or maximizing nonlinear cost functions subject to one or more constraints, such as a Levenberg-Marquardt optimizer/solver, a Nelder-Mead simplex optimizer/solver, a conjugant gradients optimizer/solver, a Gauss-Newton optimizer/solver, a Broyden-Fletcher-Goldfarb-Shanno (BFGS) optimizer/solver, a Davidon-Fletcher-Powell (DFP) optimizer/solver, a steepest descent optimizer/solver, a bisection optimizer/solver, and, when the constraints are only linear, a quadratic program solver.

9. The method of claim 7, wherein the residuals are defined as the complex valued difference between the model's predicted complex valued frequency response function H_{pred}(jw) and the observed complex valued frequency response function H_{obs}(jw).

10. The method of claim 7, wherein the residuals are defined as the difference between the model's predicted time domain output force f_{pred}(t) and the observed time domain output force data f_{obs}(t), where the input to the model is the time domain displacement data x_{obs}(t) calculated by twice integrating the observed time domain accelerance data.

11. The method of claim 7, wherein the residuals are defined as the difference between the model's predicted time domain output displacement x_{pred}(t) and the observed time domain output displacement data x_{obs}(t) calculated by twice integrating the observed time domain accelerance data, where the input to the model is the time domain force data f_{obs}(t).

12. The method of claim 1, wherein the model is a BM model.

13. The method of claim 12, wherein the BM model is a 6, 7, 8, 9, or 12 Parameter BM Model or a variant of the 6, 7, 8, 9, or 12 Parameter BM Model.

14. The method of claim 12, wherein the BM model is a finite element model of the body part that has been parameterized to 1, 2, 3, 4, 5, 6, 7, 8, 9, 10, or more parameters.

15. The method of claim 1, wherein the model is the PZ model or TF model.

16. The method of claim 1, wherein the imposed constraints include at least one constraint selected from the group consisting of: minimum and maximum system gain, system stability, minimum and maximum damping/settling time, non-negativity of physical parameters, minimum and maximum model parameter values, minimum and maximum peak frequencies, relative peak frequencies, minimum and maximum flexural rigidity, non-negativity of the quadratic radicals in Kₛ, and non-vanishing denominator in the quadratic formula for Kₛ.

17. The method of claim 1 wherein a limited number of subjects spanning the range of BMIs and muscularities are tested to determine approximate population norms for defining constraint bounds.

18. The method of claim 1, wherein (a) the subject is measured repeatedly at two or more points along one or more of a transit line in the transverse plane, a line in the longitudinal direction within the sagittal plane, different angles of the test probe relative to the cortical bone of interest and combinations therein, and (b) the results from the sweet spot, where the spurious modes of vibration are eliminated or minimized, are displayed and stored.

19. The method of claim 1, wherein (a) the subject is measured repeatedly at different points along a transit line in the transverse plane, and (b) the results from the sweet spot, where the spurious modes of vibration are eliminated or minimized, are displayed and stored.
